(19) United States
(12) Patent Application Publication
(10) Pub. No.: US 2026/0263833 A1
(43) Pub. Date: Sep. 10, 2026

(54) INTRACAVITARY APPLICATORS FOR RADIATION ONCOLOGY

(71) Applicant: University of Kentucky Research Foundation, Lexington, KY (US)

(72) Inventors: Ali Soleimani-Meigooni, Lexington, KY (US); Ellis L. Johnson, Lexington, KY (US); Denise Fabian, Lexington, KY (US)

(21) Appl. No.: 19/559,405

(22) Filed: Mar. 6, 2026

Related U.S. Application Data
(60) Provisional application No. 63/768,084, filed on Mar. 6, 2025.
(60) Provisional application No. 63/768,295, filed on Mar. 7, 2025.

(51) Int. Cl. A61N 5/10

(52) U.S. Cl. CPC A61N 5/1016

(57) ABSTRACT

An intracavitary applicator for radiation oncology includes a first arm in which a radiation source can be received and a second arm that is coupled to the first arm and in which the radiation source can be received. Each arm includes a handle that defines a proximal end of the arm and a tandem that can be inserted into a uterine cavity of an individual and defines a distal end of the arm. The handle and tandem of each arm collectively define an internal channel of the arm that can receive and guide the radiation source toward a distal end of the arm. For each arm, a portion of the internal channel defined by the tandem of the arm curves along multiple planes to account for uterine anteflexion and/or anteversion.

### RELATED APPLICATIONS

This application claims priority to U.S. Patent Application Ser. No. 63/768,084 filed on Mar. 6, 2025 and U.S. Patent Application Ser. No. 63/768,295, filed on Mar. 7, 2025 the entire disclosure of each of which is incorporated herein by reference.

### TECHNICAL FIELD

The present disclosure generally relates to intracavitary applicators for radiation oncology. In particular, embodiments of the present disclosure relate to dual-arm tandem intracavitary applicators with multi-planar curvature to account for uterine anteflexion and/or anteversion during brachytherapy treatment.

### INTRODUCTION

Brachytherapy is a treatment used to treat various types of cancers, including vaginal, cervical, uterine, and endometrial cancer. During brachytherapy, a sealed radioactive source (or radiation source) is placed inside an applicator that is implanted in the body of the patient in proximity to a tumor or an area from which a tumor was excised. Intracavitary placement of the radiation source during brachytherapy generally occurs around a natural body cavity. This technique facilitates a more targeted and concentrated dose of radiation to the cancer cells while minimizing damage to surrounding healthy tissues relative to the alternative method of treatment with external beam radiation. Brachytherapy can be utilized alone or in combination with other cancer treatment techniques, such as surgery, external beam radiotherapy and chemotherapy. Based on the location and size of the tumor, different types of applicators have been designed and gradually modified to improve quality treatments.

Hysterectomy is a treatment for endometrial cancer. However, in some cases, a patient is not a candidate for hysterectomy due to medical comorbidities or other issues which render the patient a poor candidate for surgery. In such cases, radiation is used as a curative option for inoperable endometrial cancer, with brachytherapy being critical for effective treatment. Brachytherapy for inoperable endometrial cancer is typically done utilizing a Y-tandem applicator that provides two channels in which a radiation source is deposited. The apex/tip of each channel of the Y-tandem applicator are placed at one uterine cornu to facilitate dose distribution to the entirety of the uterus.

However, existing Y-tandem applicators of known construction generally fail to adequately dose the uterus in most patients because: (1) the channel length is typically too short for most women requiring brachytherapy; and/or (2) the channels only possess a single curvature that partially facilitates lateral curvature into the cornu and does not match the anatomy of patients. With respect to the former, most Y-tandem applicators include channels which have a length that extends only 60% of that of the length of the uterus of some patients. With respect to the latter, the single curvature possessed by the channels neglect the anatomic anteflexion and anteversion of the uterus, which ultimately leads to underdosing of the portion of the uterus that is reflected anteriorly. As a result, the isodose lines resulting from Y-tandem applicators of known construction do not match the target anatomy of the patient. Another problem with Y-tandem applicators of known construction is that the arms of the applicator defining the channel occupy a space which is typically wider than the patient's anatomy when in a non-compressed configuration. Accordingly, the arms of the applicator will typically collapse towards each other as a result of the compressive force imposed on the applicator by the patient's anatomy, which can prevent the Y-tandem applicator from dosing the patient with radiation in the desired manner.

### SUMMARY

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The present disclosure includes intracavitary applicators useful in radiation oncology applications, such as brachytherapy treatment. In particular, the present disclosure includes intracavitary applicators in the form of dual-arm tandem applicators with multi-planar curvature to account for uterine anteflexion and/or anteversion.

An exemplary intracavitary applicator for radiation oncology includes: a first arm in which a radiation source can be received; and a second arm that is coupled to the first arm and in which the radiation source can be received. The first arm includes: a first handle that defines a proximal end of the first arm; and a first tandem that defines the distal end of the first arm and is configured to be inserted in a uterine cavity of an individual. The first handle and the first tandem collectively define a first internal channel with an open proximal end and a closed distal end. The first internal channel can receive and guide the radiation source from the proximal end of the first arm toward the distal end of the first arm. The second arm includes: a second handle that defines a proximal end of the second arm; and a second tandem that defines the distal end of the second arm and is configured to be inserted in the uterine cavity of the individual. The second handle and the second tandem collectively define a second internal channel with an open proximal end and a closed distal end. The second internal channel can receive and guide the radiation source from the proximal end of the second arm toward the distal end of the second arm. A portion of the first internal channel defined by the first tandem and a second internal channel defined by the second tandem each curve along multiple planes.

In some embodiments, the portion of the first internal channel defined by the first tandem and the portion of the second internal channel defined by the second tandem curving along multiple planes each curve along two planes that are perpendicular to each other. In some embodiments, the portion of the first internal channel defined by the first tandem and the portion of the second internal channel defined by the second tandem are each oriented to curve along a sagittal plane and a coronal plane when the first tandem and the second tandem are inserted in the uterine cavity of the individual.

In some embodiments, a first portion of the first tandem and a first portion of the second tandem are oriented at an angle ranging from about 10 degrees to about 30 degrees relative to the first handle and the second handle, respectively. In some embodiments, a second portion of the first tandem and a second portion of the second tandem are oriented at an angle ranging from about 10 degrees to about 15 degrees relative to the first handle and the second handle, respectively. In some embodiments, the first tandem and the second tandem each include a first curved portion and a second curved portion. In some embodiments, the first tandem and the second tandem are shaped, such that, the second curved portion of the first tandem and the second curved portion of the second tandem curve along a common plane in opposite directions when the first curved portion of the first tandem and the first curved portion of the second tandem are oriented to curve in the same direction along parallel planes.

In some embodiments, the intracavitary applicator further includes a coupler for coupling the first arm and the second arm. In various embodiments, the coupler can include a guide and/or a locking device. The guide includes a first internal channel in which the first arm is slidably received and a second internal channel in which the second arm is slidably received. In some embodiments, the guide further includes a third internal channel that is configured to receive the radiation source. In some embodiments, the third internal channel forms a ring. In some embodiments, the applicator can further include a conduit that is attached to the guide and is configured to direct the radiation source from outside of the guide into the third channel. In some embodiments, the guide has a diameter ranging from about 2.0 centimeters to about 3.0 centimeters. The locking device can be selectively manipulated to hold the first arm and the second arm in a fixed position.

In some embodiments, the first tandem and the second tandem each have a length ranging from about 6 centimeters to about 13 centimeters. In various embodiments, the diameter of the first arm and/or the second arm can be substantially the same or vary along its length.

### BRIEF DESCRIPTION OF THE DRAWINGS

FIG. 1 is a perspective view of an intracavitary applicator made in accordance with an embodiment of the present disclosure;

FIG. 2 is a side view of the intracavitary applicator of FIG. 1;

FIG. 3 is a top view of the intracavitary applicator of FIG. 1;

FIG. 4 is a perspective view of a first arm and a second arm of the intracavitary applicator of FIG. 2;

FIG. 5 is a perspective view of a guide of the intracavitary applicator of FIG. 1;

FIG. 6 is a perspective view of an intracavitary applicator made in accordance with another embodiment of the present disclosure;

FIG. 7 is a top view of the intracavitary applicator of FIG. 6;

FIG. 8 is a perspective view of a guide of the intracavitary applicator of FIG. 6;

FIG. 9 is a cross-sectional view of the guide taken along 9-9 in FIG. 8; and

FIG. 10 is a side view of an embodiment of an arm that can be utilized in an intracavitary applicator made in accordance with the present disclosure.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly, the specific details of the described exemplary embodiments, is provided primarily for clearness of understanding and no unnecessary limitations are to be understood therefrom. In case of conflict, the specification of this document, including definitions, will control.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Unless otherwise indicated, all numbers expressing quantities of dimension and angle used in the specification are to be understood as being modified in all instances by the term “about”. Accordingly, unless indicated to the contrary, the numerical parameters set forth in this specification are approximations that can vary depending upon the desired properties sought to be obtained by the presently disclosed subject matter.

As used herein, the term “about,” when referring to a value or to dimension or angle is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

It is understood that where a range of units is disclosed, such range is inclusive of the starting and end units. For example, if a range of “10 to 15” or “between 10 to 15” is disclosed, then 10 and 15 are considered part of such range, unless stated otherwise. It is also understood that each unit between two particular units are also disclosed. For example, if 10 and 15 are disclosed, then 11, 12, 13, and 14 are also disclosed.

The present disclosure includes intracavitary applicators. In particular, certain embodiments of the present disclosure are directed to dual-arm tandem applicators with multi-planar tandem curvature to account for uterine anteflexion and anteversion during brachytherapy treatment. As will be appreciated by one of ordinary skill in the art upon study of this document, intracavitary applicator embodiments disclosed herein can be useful during brachytherapy treatment of an individual, such as a human born biologically female. Cancers for which brachytherapy treatment can be applied as treatment include, by way of non-limiting example, vaginal, cervical, uterine, and endometrial cancer.

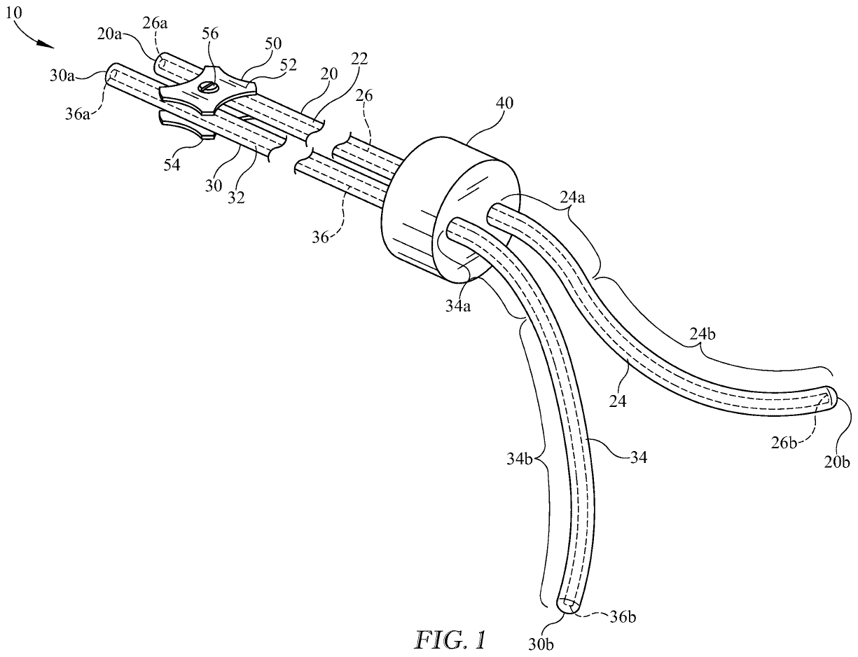

Referring first to FIGS. 1-3, an exemplary intracavitary applicator in the form of a dual-arm tandem applicator (or applicator) 10 is provided and includes: a first arm 20; and a second arm 30 that is coupled to the first arm 20. As the applicators disclosed herein include two tandems, the disclosed applicators may also be characterized as “double-tandem applicators”. The first arm 20 includes a handle portion (or first handle) 22 that defines the proximal end 20a of the first arm and a tandem portion (or first tandem) 24 that defines the distal end 20b of the first arm 20 and is configured to be inserted into the vagina, through the cervical canal, and into the uterine cavity of an individual. The second arm 30 likewise includes a handle portion (or second handle) 32 that defines the proximal end 30a of the second arm 30 and a tandem portion (or second tandem) 34 that defines the distal end 30b of the second arm and is configured to be inserted into the vagina, through the cervical canal, and into the uterine cavity of the individual. As shown, the distal ends 20b, 30b of the first arm 20 and the second arm 30 can be rounded to facilitate intracavitary insertion of the first tandem 24 and the second tandem 24.

Referring now specifically to FIG. 1, to facilitate the guidance of a radiation source into the uterine cavity of the individual for radiation treatment when the applicator 10 is in use, the first arm 20 and the second arm 30 each include an internal channel 26, 36 in which the radiation source can be deposited and travel along a length of the first arm 20 and the second arm 30. In this regard, the first handle 22 and the first tandem 24 collectively define an internal channel (or first internal channel) 26 that includes an open proximal end 26a located at the proximal end 20a of the first arm 20 and a closed distal end 26b located proximate to the distal end 20b of the first arm 20. Likewise, the second handle 32 and the second tandem 34 collectively define an internal channel (or second internal channel) 36 in the second arm 30 that includes an open proximal end 36a located at the proximal end 30a of the first arm 30 and a closed distal end 36b located proximate to the distal end 30b of the second arm 30.

In use, the applicator 10 can be utilized to apply brachytherapy treatment for uterine cancer by depositing the applicator 10 in an individual so that the first tandem 24 and the second tandem 34, or a portion thereof, is inserted in a uterine cavity of the individual. Once the applicator 10 is deposited in the individual, a radiation source (not shown) can be sequentially inserted into the first internal channel 26 and the second internal channel 36 to apply radiation dosing to the target treatment area.

The radiation source can be moved to different locations along the first internal channel 26 and the second internal channel 36 to apply radiation dosing to different areas of the individual's anatomy. In this regard, during brachytherapy, such as high-dose radiation (HDR) brachytherapy, the radiation source can be attached to the end of a wire and can be selectively deposited in the first internal channel 26 and the second internal channel 36 at different positions therein to facilitate treatment of an individual. Treatment planning software can be utilized to determine the positions in the first internal channel 26 and the second internal channel 36 that the radiation source will stop for a given period of time to deliver the required radiation prior to moving to a different location in the channel for a subsequent period of time at such location, and so on. These positions are known as “dwell” positions and the time that the radiation source remains in that position is known as “dwell” time. The radiation source and/or dwell positions along each internal channel 26, 36 while the applicator 10 is in use can be determined based on the known length of the radiation source. In some embodiments, each respective internal channel 26, 36 is configured to accommodate multiple dwell positions per centimeter of the treatment length (i.e., length of a selected section of the patient's anatomy targeted for treatment).

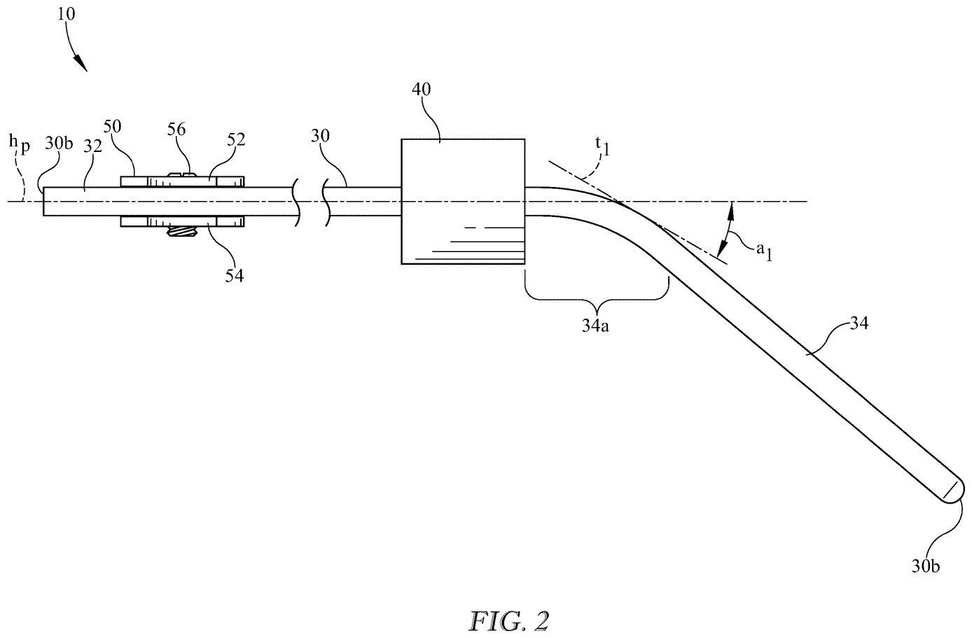

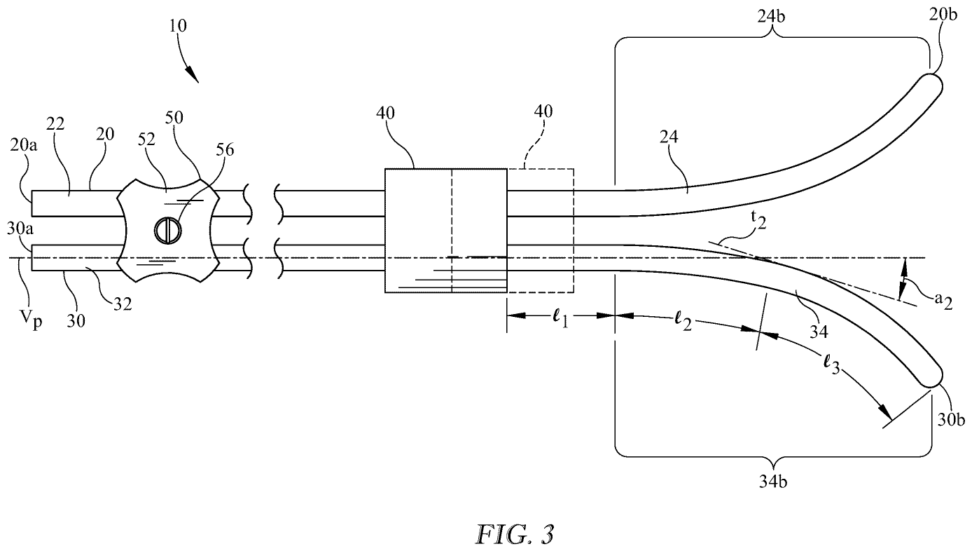

Referring now again to FIGS. 1-3, the first tandem 24 and the second tandem 34 are each shaped to curve along multiple planes. Accordingly, a portion of the first internal channel 26 defined by the first tandem 24 and a portion of the second internal channel 36 defined by the second tandem 34 likewise curve along multiple planes, as best shown in FIG. 1. As evidenced by comparing FIGS. 2 and 3, in this exemplary embodiment, the first tandem 24 and the second tandem 34, and thus the portions of the first internal channel 26 and the second internal channel 36 defined thereby, curve along a first plane and a second plane that are perpendicular to each other. In this regard, the first tandem 24 and the second tandem 34 each include a first curved portion 24a, 34a and a second curved portion 24b, 34b that cause the first tandem 24 and the second tandem 34 to exhibit curvature along a first plane and a second plane, respectively, that are perpendicularly oriented to each other.

Referring still to FIGS. 1-3, when the applicator 10 is in use, the first tandem 24 and the second tandem 34 can be oriented and deposited in an individual in need of brachytherapy treatment in a manner that better accounts for uterine anteversion and/or anteflexion in the individual and provides more effective radiation dosing to portions of the uterus reflected anteriorly than traditional Y-tandem applicators. Specifically, the first tandem 24 and the second tandem 34 can be oriented, such that the curvature exhibited by the first curved portion 24a of the first tandem 24 and the curvature exhibited by the first curved portion 34a of the second tandem 34 each occur along a sagittal plane and towards the anterior of the individual receiving treatment, as perhaps best reflected in FIG. 2. As the first tandem 24 and the second tandem 34 exhibit curvature along two perpendicularly oriented planes, when the first tandem 24 and the second tandem 34 are so oriented, the second curved portion 24b of the first tandem 24 and the second curved portion 34b of the second tandem 34 will curve along a coronal plane of the individual receiving treatment, as perhaps best reflected in FIG. 3. Curvature along the coronal plane facilitates deposit of the distal end 20b of the first arm 20 and the distal end 30b of the second arm 30 in a uterine cornu of the individual. In this exemplary embodiment, the first tandem 24 and the second tandem 34 are shaped, such that, when the first curved portion 24a of the first tandem 24 and the first curved portion 34a of the second tandem 34 are oriented as to curve along two sagittal planes of the individual in the same direction, the second curved portion 24b of the first tandem 24 and the second curved portion 34b of the second tandem 34 curve along a common coronal plane of the individual in opposite directions, as best shown in FIG. 3. In other words, when the first curved portion 24a of the first tandem 24 and the first curved portion 34a of the second tandem 34 are oriented as to curve in the same direction along parallel planes, the second curved portion 24b of the first tandem 24 and the second curved portion 34b of the second tandem 34 curve in opposite directions along a common plane. In this way, the first curved portions 24a, 34a and the second curved portions 24b, 34b of the first tandem 24 and the second tandem 34 enable the applicator 10 to account for uterine anteversion and/or anteflexion while still facilitating tandem placement, and thus dose distribution, at both uterine cornus of the individual receiving treatment.

Referring now specifically to FIG. 2, as evidenced by the intersection of tangent plane, t₁, that is tangent to the first curved portion 34a of the second tandem 34 and a horizontal plane, hp, along which a centerline of a linear segment of the second handle 32 extends, a first portion of the second tandem 34 is angled relative to the second handle 32 of the second arm 30, as indicated by angle, a₁. A first portion of the first tandem 24 is angled relative to the first handle 22 of the first arm 20 in the same manner as the first portion of the second tandem 34 is angled relative to the second handle 32. In order to identify the orientation of the applicator within an individual, some dual-tandem applicators have an unnecessary curvature on the handle that causes the collapsing of the applicator as the legs of an individual receiving brachytherapy treatment are straightened. To further address this issue, in some embodiments, the entirety of the first handle 22 and the second handle 32 is linear. The curvature of the first curved portion 24a, 34a of the first tandem 24 and/or the second tandem 34 can be adjusted to accommodate the degree of uterine anteversion and/or uterine anteflexion in different individuals, and thus the extent to which the first portion of first tandem 24 and the second tandem 34 are angled relative to the first handle 22 and the second handle 32, respectively. In some embodiments, the first curved portion 24a of the first tandem 24 and the first curved portion 34a of the second tandem 34 are curved, such that a first portion of the first tandem 24 and a first portion of the second tandem 34 are oriented at an angle from about 10 degrees to about 30 degrees relative to the first handle 22 and the second handle 32, respectively.

Referring now specifically to FIG. 3, as evidenced by the intersection of tangent plane, t₂, that is tangent to the second curved portion 34b of the second tandem 34 and a vertical plane, vₚ, along which a centerline of a linear segment of the second handle 34 extends, a second portion of the second tandem 34 is angled relative to the second handle 32 of the second arm 30, as indicated by angle, a₂. A second portion of the first tandem 24 is similarly angled relative to the first handle 22 of the first arm 20. The curvature of the second curved portion 24b, 34b of the first tandem 24 and/or the second tandem 34 can be adjusted to accommodate the degree of spacing between uterine cornu in different individuals, and thus the extent to which the second portion of the first tandem 24 and the second tandem 34 are angled relative to the first handle 22 and the second handle 32, respectively. In some embodiments, the second curved portion 24b of the first tandem 24 and the second curved portion 34b of the second tandem 34 are curved, such that a second portion of the first tandem 24 and a second portion of the second tandem 34 are oriented at an angle from about 10 degrees to about 15 degrees relative to the first handle 22 and the second handle 32, respectively.

The length of the first tandem 24 and the second tandem 34 can also be adjusted to accommodate the anatomy of different individuals. In some embodiments, the first tandem 24 and the second tandem 34 each have a length of about 6 centimeters (cm) to about 13 cm. In some embodiments, the first arm 20 and the second arm 30 each have a total length, corresponding to the combined length of the handle and the tandem, of about 32 cm. Of course, the total length of the first arm 20 and the total length of the second arm 30 can be increased or decreased (e.g., by increasing or decreasing the handle length) without departing from the spirit and scope of the present disclosure.

Referring now again to FIG. 3, in this exemplary embodiment, the length of the second tandem 34 is indicated by the combined length of l₁, l₂, and l₃, where l₁ is the length of the first curved portion 34a of the second tandem 34 and l₂ and l₃, correspond to different segments of the second curved portion 34b of the second tandem 34. Specifically, in this exemplary embodiment, l₃ corresponds to a more gradually curved segment of the second curved portion 34b and l₂ corresponds to a less gradually curved segment of the second curved portion 34b of the second tandem 34. In some embodiments, the length of l₁ ranges from about 2 cm to about 5 cm, the length of l₂ ranges from about 3 cm to about 5 cm, and the length of l₃ ranges from about 1 cm to about 3 cm. It is appreciated that while l₃ is primarily illustrated in the drawings and described herein in the context of corresponding to the second curved portion 34b, alternative embodiments in which l₃ corresponds to the first curved portion 34a of second tandem 34 are also contemplated herein, as are embodiments in which l₃ corresponds to a portion of the tandem which is not curved and separates two curved portions of the second tandem 34. In the exemplary applicator 10 embodiment shown in FIGS. 1-3, the first arm 20 and the second arm 30 are of identical construction, except that the second curved portion 24b of the first tandem 24 and the second curved portion 34b of the second tandem 34 curve in opposite directions of each other. Accordingly, the above discussion regarding the length of the second tandem 34 of the second arm 30 is also applicable to the length of the first tandem 24 of the first arm 20.

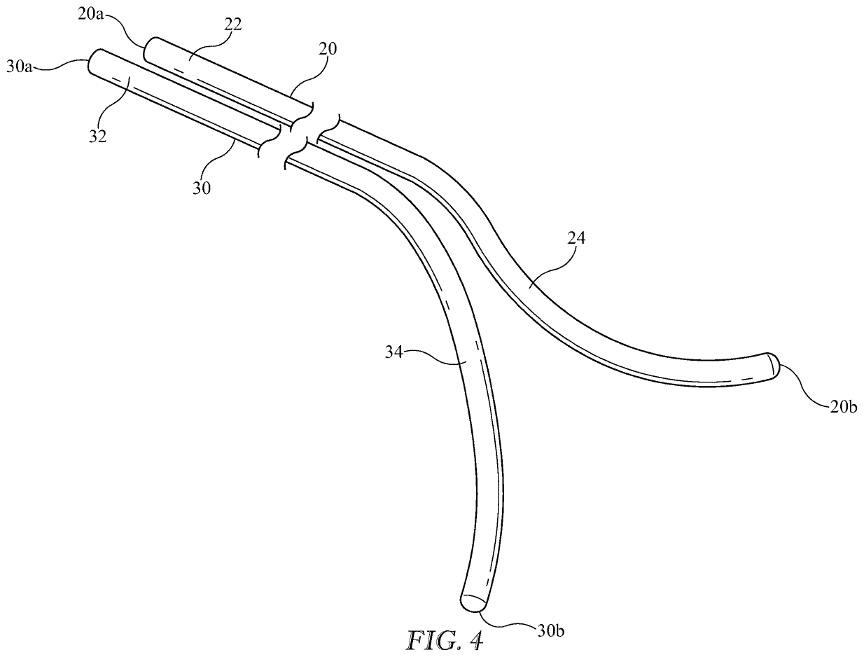

Referring now to FIGS. 1 and 4, in this embodiment, the first arm 20 and the second arm 30 are each integrally formed, such that the handle 22, 32 and the tandem 24, 34 of each respective arm 20, 30 is formed by a unitary member. Alternative embodiments in which the first handle 22 and the first tandem 24 of the first arm 20 are separate members joined together and/or in which the second handle 32 and second tandem 34 of the second arm 30 are separate members joined together are, however, also contemplated herein. As shown best in FIG. 4, in this embodiment, the outer diameter of the handle 22, 32 and the tandem 24, 34 of each respective arm 20, 30 are substantially the same. In some embodiments, the outer diameter of the handle 22, 32 and the tandem 24, 34 of each respective arm 20, 30 is about 3 millimeters (mm) to about 6 mm. As best shown in FIG. 1, in this embodiment, the first internal channel 26 is of the same diameter throughout the length of the first arm 20, and the second internal channel 36 is of the same diameter throughout the length of the second arm 30. In some embodiments, the diameter of the first internal channel 26 and the diameter of the second internal channel 36 is about 1.5 mm to minimize the curvilinear path of source wires transporting the radiation source within the first internal channel 26 and the second internal channel 36.

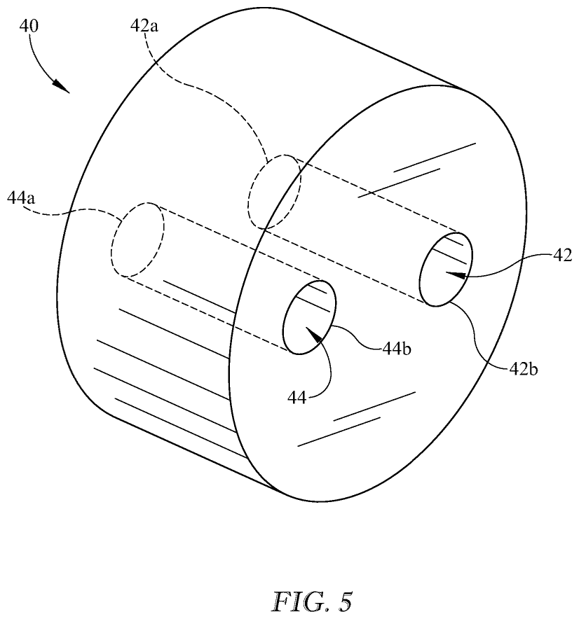

Referring now to FIGS. 1-3 and 5, the first arm 20 and the second arm 30 are coupled by a coupler that, in this exemplary embodiment, includes both a guide 40 and a locking device 50. The guide 40 includes: a first internal channel 42 with two open ends 42a, 42b and in which the first arm 20 is received; and a second internal channel 44 with two open ends 44a, 44b and in which the second arm 30 is received. The first internal channel 42 and the second internal channel 44 of the guide 40 are spaced apart from each other to maintain the portion of the first arm 20 and the second arm 30 received in the first internal channel 42 and the second internal channel 44, respectively, a predetermined distance from each other while the device is in use.

Referring still to FIGS. 1-3 and 5, during brachytherapy treatment, the applicator 10 can, in some implementations, be positioned so that the guide 40 abuts or is positioned proximal to the external OS of the cervix of an individual. In some implementations, vaginal packing materials may be utilized to provide spacing between the guide 40 and the external OS of the cervix of the individual. As shown best in FIG. 3, in this embodiment, the first arm 20 and the second arm 30 are slidably received in the first internal channel 42 and the second internal channel 44, respectively, of the guide 40. Specifically, the first arm 20 and the second arm 30 are slidably received, such that the first arm 20 and the second arm 30 can travel within the first internal channel 42 and the second internal channel 44 of the guide 40. Through such travel, the extent to which the first tandem 24 and the second tandem 34 are deposited in the cervical canal and uterine cavity of the individual can be affected when the applicator 10 is in use. In this exemplary embodiment, the first arm 20 and the second arm 30 can be rotated while deposited in the first channel 42 and the second channel 44 of the guide 40 so that the orientation of the first tandem 24 and the second tandem 34 can be adjusted by rotating the first arm 20 and the second arm 30, respectively. Accordingly, in this exemplary embodiment, the first arm 20 and the second arm 30 can be selectively manipulated to cause the first tandem 24 and the second tandem 34 to assume various orientations and positions to facilitate treatment of different target treatment areas in an individual.

Referring still to FIGS. 1-3 and 5, in some embodiments, the diameter of the first arm 20 is substantially the same as the diameter of the first internal channel 42 of the guide 40, such that the two can form an interface fit that can be overcome with the application of a pushing force to the first handle 22 to affect the positioning of the guide 40 along the length of the first arm 20 and/or with the application of a rotational force to the first handle 22 to affect the orientation of the first tandem 24. Likewise, in some embodiments, the diameter of the second arm 30 is substantially the same as the diameter of the second internal channel 44 of the guide 40, such that the two can form an interface fit that can be overcome with the application of a pushing force to the second handle 32 to affect the positioning of the guide 40 along the length of the second arm 30 and/or with the application of a rotational force to the second handle 32 to affect the orientation of the second tandem 34.

Referring still to FIGS. 1-3 and 5, in this exemplary embodiment, the guide 40 is in the cylindrically shaped and has a diameter of about 2.0 cm to about 3.0 centimeters. The rounded contours provided by a cylindrically shaped guide 40 conforms well to vaginal cavity in which it will typically be positioned when the applicator 10 is in use, and thus may serve to reduce individual discomfort during brachytherapy treatment as compared to other shapes. However, alternative embodiments in which the shape and/or diameter of the guide 40 are adjusted to accommodate different anatomical environments and/or brachytherapy applications are also contemplated herein.

Referring now again to FIGS. 1-3, the locking device 50 is configured to hold the first arm 20 and the second arm 30 in a fixed position as to prevent rotation or shift of the first arm 20 and the second arm 30 once they have been properly placed in an individual. In this exemplary embodiment, the locking device 50 is in the form of a clamp-style fastener that maintains the first arm 20 and the second arm 30 in a fixed position by applying a clamping force thereto. In this regard, the locking device 50 includes a pair of opposing plates 52, 54 between which the first arm 20 and the second arm 30 are deposited, and a screw 56. The screw 56 can be selectively tightened to draw the opposing plates 52, 54 closer together to hold the first arm 20 and the second arm 30 in a fixed position. Conversely, the screw 56 can be loosened to move the opposing plates 52, 54 further apart to permit rotation and/or sliding of the first arm 20 and the second arm 30 within the guide 40. In alternative embodiments, other locking devices that can be selectively manipulated by a user to transition the locking device between a first state that holds the first arm 20 and the second arm 30 in a fixed position and a second state that permits rotation and/or sliding of the first arm 20 and the second arm 30 in the guide 40 can be utilized without departing from the spirit and scope of the present disclosure. In some embodiments and implementations, the first arm 20 and the second arm 30 can be held in a fixed position by the locking device 50, such that the first tandem 24 and the second tandem 34 each curve along both a sagittal and a coronal plane when inserted into the uterine cavity of an individual.

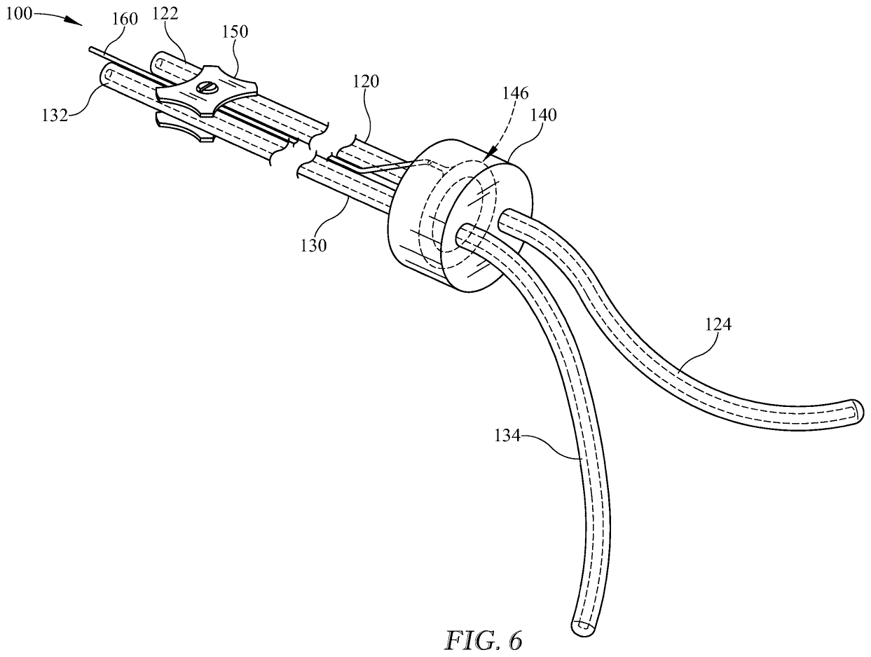

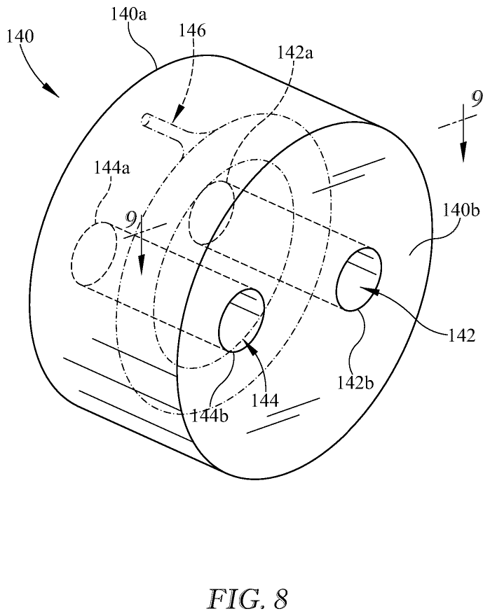

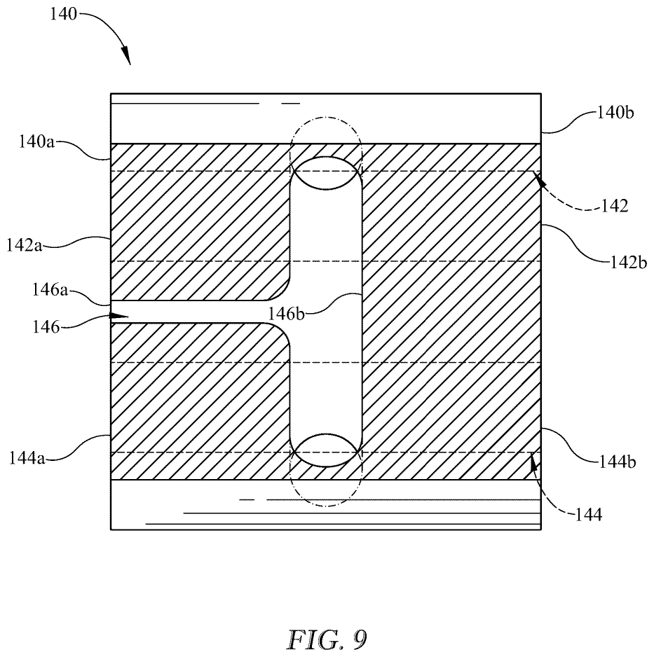

Referring now to FIGS. 6-9, another exemplary embodiment of an intracavitary applicator in the form of a dual-arm tandem applicator (or applicator) 100 is provided and includes: a first arm 120; a second arm 130; a guide 140; and a locking device 150. The first arm 120, the second arm 130, the guide 140 and the locking device 150 in this exemplary embodiment are of identical construction and function in the same manner as the first arm 20, the second arm 30, the guide 40 and the locking device 50, respectively, of the applicator 10 described above with reference to FIGS. 1-5, except as otherwise noted. Like the guide 40 of the applicator 10 described above with reference to FIGS. 1-5, in this exemplary embodiment, the guide 140 also includes: a first internal channel 142 with two open ends 142a, 142b and in which the first arm 120 is received; and a second internal channel 144 with two open ends 144a, 144b and in which the second arm 130 is received. However, unlike the guide 40 of the applicator 10 described above with reference to FIGS. 1-5, in this exemplary embodiment, the guide 140 further includes a third internal channel 146 with an open proximal end 146a and a closed distal end 146b that forms a ring which encircles the first internal channel 142 and the second internal channel 144. The third internal channel 146 is configured to receive and guide the radiation source from a proximal end 140a of the guide 140 around the first internal channel 142 and the second internal channel 144. When the applicator 100 is in use, the distal end 140b of the guide 140 can be positioned as to abut or be proximally located to the external OS of the cervix of an individual and the radiation source can be deposited in the third internal channel 146 of the guide 140 to apply radiation dosing to the cervix of the individual. Accordingly, the guide 140 in this exemplary embodiment can, in some implementations, effectively act as a brachytherapy ring applicator when the applicator 100 is in use. The applicator 100 of this exemplary embodiment may thus find particular utility with respect to brachytherapy treatment of individuals with both endometrial and cervical cancer.

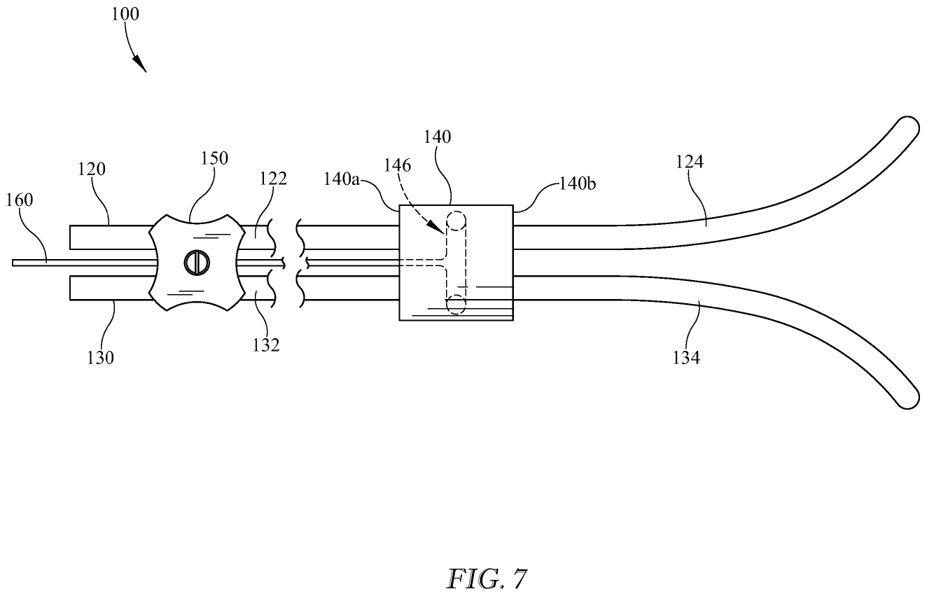

Referring now specifically to FIGS. 6 and 7, to facilitate deposit of the radiation source into the third internal channel 146 of the guide 140, the applicator 10 can further include a conduit 160 attached to the guide 140. The conduit 160 includes an internal channel (not shown) that enables travel of the radiation source through the conduit 160. In some embodiments, a distal end of the conduit 160 can be deposited in the third internal channel 146 of the guide 140, such that, when the radiation source is located in the distal end of the conduit 160 it is also provided in the third internal channel 146 of the guide 140. In some embodiments, the internal channel of the conduit 160 includes two open ends, such that the distal end of the conduit 160 can be aligned with the open proximal end of the third internal channel 146 and the radiation source can travel outside of the conduit 160 and into the third internal channel 146 of the guide 140. As shown, in some embodiments, in addition to the first arm 120 and the second arm 130, the locking device 150 can also receive the conduit 160 facilitating deposit of the radiation source into the third internal channel 146 of the guide 140. Alternative embodiments, in which the conduit 160 is not received by the locking device 150 are, however, also contemplated herein.

As reflected in TABLES 1-8 below, various embodiments of the applicators 10, 100 can be established by modifying: the diameter of the guide 40, 140; the angle, a₁, between the handles 22, 32, 122, 132 and a first portion of the tandems 24, 34, 124, 134 of the first arm 20, 120 and the second arm 30, 130; the angle, a₂, between the handles 22, 32, 122, 132 and the second portion of the tandems 24, 34, 124, 134 of the first arm 20, 120 and the second arm 30, 130; the total length of the tandems 24, 34, 124, 134 of the first arm 20, 120 and the second arm 30, 130; and/or the lengths of different portions, l₁, l₂, l₃, of the tandems 24, 34, 124, 134 of the first arm 20, 120 and the second arm 30, 130.

TABLE 1
Tandem Length (l₁ + l₂ + l₃) = 6 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1A | 2.5 | 10 | 10 | 2 | 3 | 1 | 32 |
| 2A | 2.5 | 10 | 15 | 2 | 3 | 1 | 32 |
| 3A | 2.5 | 15 | 10 | 2 | 3 | 1 | 32 |
| 4A | 2.5 | 15 | 15 | 2 | 3 | 1 | 32 |
| 5A | 2.5 | 30 | 10 | 2 | 3 | 1 | 32 |
| 6A | 2.5 | 30 | 15 | 2 | 3 | 1 | 32 |
| 7A | 3.0 | 10 | 10 | 2 | 3 | 1 | 32 |
| 8A | 3.0 | 10 | 15 | 2 | 3 | 1 | 32 |
| 9A | 2.0 | 15 | 10 | 2 | 3 | 1 | 32 |
| 10A | 3.0 | 15 | 15 | 2 | 3 | 1 | 32 |
| 11A | 3.0 | 30 | 10 | 2 | 3 | 1 | 32 |
| 12A | 3.0 | 30 | 15 | 2 | 3 | 1 | 32 |

TABLE 2
Tandem Length (l₁ + l₂ + l₃) = 7 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1B | 2.5 | 10 | 10 | 3 | 3 | 1 | 32 |
| 2B | 2.5 | 10 | 15 | 3 | 3 | 1 | 32 |
| 3B | 2.5 | 15 | 10 | 3 | 3 | 1 | 32 |
| 4B | 2.5 | 15 | 15 | 3 | 3 | 1 | 32 |
| 5B | 2.5 | 30 | 10 | 3 | 3 | 1 | 32 |
| 6B | 2.5 | 30 | 15 | 3 | 3 | 1 | 32 |
| 7B | 3.0 | 10 | 10 | 3 | 3 | 1 | 32 |
| 8B | 3.0 | 10 | 15 | 3 | 3 | 1 | 32 |
| 9B | 3.0 | 15 | 10 | 3 | 3 | 1 | 32 |
| 10B | 3.0 | 15 | 15 | 3 | 3 | 1 | 32 |
| 11B | 3.0 | 30 | 10 | 3 | 3 | 1 | 32 |
| 12B | 3.0 | 30 | 15 | 3 | 3 | 1 | 32 |

TABLE 3
Tandem Length (l₁ + l₂ + l₃) = 8 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1C | 2.5 | 10 | 10 | 3 | 3 | 2 | 32 |
| 2C | 2.5 | 10 | 15 | 3 | 3 | 2 | 32 |
| 3C | 2.5 | 15 | 10 | 3 | 3 | 2 | 32 |
| 4C | 2.5 | 15 | 15 | 3 | 3 | 2 | 32 |
| 5C | 2.5 | 30 | 10 | 3 | 3 | 2 | 32 |
| 6C | 2.5 | 30 | 15 | 3 | 3 | 2 | 32 |
| 7C | 3.0 | 10 | 10 | 3 | 3 | 2 | 32 |
| 8C | 3.0 | 10 | 15 | 3 | 3 | 2 | 32 |
| 90 | 3.0 | 15 | 10 | 3 | 3 | 2 | 32 |
| 10C | 3.0 | 15 | 15 | 3 | 3 | 2 | 32 |
| 11C | 3.0 | 30 | 10 | 3 | 3 | 2 | 32 |
| 12C | 3.0 | 30 | 15 | 3 | 3 | 2 | 32 |

TABLE 4
Tandem Length (l₁ + l₂ + l₃) = 9 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1D | 2.5 | 10 | 10 | 3 | 4 | 2 | 32 |
| 2D | 2.5 | 10 | 15 | 3 | 4 | 2 | 32 |
| 3D | 2.5 | 15 | 10 | 3 | 4 | 2 | 32 |
| 4D | 2.5 | 15 | 15 | 3 | 4 | 2 | 32 |
| 5D | 2.5 | 30 | 10 | 3 | 4 | 2 | 32 |
| 6D | 2.5 | 30 | 15 | 3 | 4 | 2 | 32 |
| 7D | 3.0 | 10 | 10 | 3 | 4 | 2 | 32 |
| 8D | 3.0 | 10 | 15 | 3 | 4 | 2 | 32 |
| 9D | 3.0 | 15 | 10 | 3 | 4 | 2 | 32 |
| 10D | 3.0 | 15 | 15 | 3 | 4 | 2 | 32 |
| 11D | 3.0 | 30 | 10 | 3 | 4 | 2 | 32 |
| 12D | 3.0 | 30 | 15 | 3 | 4 | 2 | 32 |

TABLE 5
Tandem Length (l₁ + l₂ + l₃) = 10 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1E | 2.5 | 10 | 10 | 4 | 4 | 2 | 32 |
| 2E | 2.5 | 10 | 15 | 4 | 4 | 2 | 32 |
| 3E | 2.5 | 15 | 10 | 4 | 4 | 2 | 32 |
| 4E | 2.5 | 15 | 15 | 4 | 4 | 2 | 32 |
| 5E | 2.5 | 30 | 10 | 4 | 4 | 2 | 32 |
| 6E | 2.5 | 30 | 15 | 4 | 4 | 2 | 32 |
| 7E | 3.0 | 10 | 10 | 4 | 4 | 2 | 32 |
| 8E | 3.0 | 10 | 15 | 4 | 4 | 2 | 32 |
| 9E | 3.0 | 15 | 10 | 4 | 4 | 2 | 32 |
| 10E | 3.0 | 15 | 15 | 4 | 4 | 2 | 32 |
| 11E | 3.0 | 30 | 10 | 4 | 4 | 2 | 32 |
| 12E | 3.0 | 30 | 15 | 4 | 4 | 2 | 32 |

TABLE 6
Tandem Length (l₁ + l₂ + l₃) = 11 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1F | 2.5 | 10 | 10 | 4 | 5 | 2 | 32 |
| 2F | 2.5 | 10 | 15 | 4 | 5 | 2 | 32 |
| 3F | 2.5 | 15 | 10 | 4 | 5 | 2 | 32 |
| 4F | 2.5 | 15 | 15 | 4 | 5 | 2 | 32 |
| 5F | 2.5 | 30 | 10 | 4 | 5 | 2 | 32 |
| 6F | 2.5 | 30 | 15 | 4 | 5 | 2 | 32 |
| 7F | 3.0 | 10 | 10 | 4 | 5 | 2 | 32 |
| 8F | 3.0 | 10 | 15 | 4 | 5 | 2 | 32 |
| 9F | 3.0 | 15 | 10 | 4 | 5 | 2 | 32 |
| 10F | 3.0 | 15 | 15 | 4 | 5 | 2 | 32 |
| 11F | 3.0 | 30 | 10 | 4 | 5 | 2 | 32 |
| 12F | 3.0 | 30 | 15 | 4 | 5 | 2 | 32 |

TABLE 7
Tandem Length (l₁ + l₂ + l₃) = 12 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1G | 2.5 | 10 | 10 | 4 | 5 | 3 | 32 |
| 2G | 2.5 | 10 | 15 | 4 | 5 | 3 | 32 |
| 3G | 2.5 | 15 | 10 | 4 | 5 | 3 | 32 |
| 4G | 2.5 | 15 | 15 | 4 | 5 | 3 | 32 |
| 5G | 2.5 | 30 | 10 | 4 | 5 | 3 | 32 |
| 6G | 2.5 | 30 | 15 | 4 | 5 | 3 | 32 |
| 7G | 3.0 | 10 | 10 | 4 | 5 | 3 | 32 |
| 8G | 3.0 | 10 | 15 | 4 | 5 | 3 | 32 |
| 9G | 3.0 | 15 | 10 | 4 | 5 | 3 | 32 |
| 10G | 3.0 | 15 | 15 | 4 | 5 | 3 | 32 |
| 11G | 3.0 | 30 | 10 | 4 | 5 | 3 | 32 |
| 12G | 3.0 | 30 | 15 | 4 | 5 | 3 | 32 |

TABLE 8
Tandem Length (l₁ + l₂ + l₃) = 13 cm

| | | | | | | | Total |
|---|---|---|---|---|---|---|---|
| | Diam- | | | | | | Length |
| Appli- | eter of | | | | | | (Tandem + |
| cator | Guide | a₁ | a₂ | l₁ | l₂ | l₃ | Handle) |
| # | (cm) | (degree) | (degree) | (cm) | (cm) | (cm) | (cm) |
| 1H | 2.5 | 10 | 10 | 5 | 5 | 3 | 32 |
| 2H | 2.5 | 10 | 15 | 5 | 5 | 3 | 32 |
| 3H | 2.5 | 15 | 10 | 5 | 5 | 3 | 32 |
| 4H | 2.5 | 15 | 15 | 5 | 5 | 3 | 32 |
| 5H | 2.5 | 30 | 10 | 5 | 5 | 3 | 32 |
| 6H | 2.5 | 30 | 15 | 5 | 5 | 3 | 32 |
| 7H | 3.0 | 10 | 10 | 5 | 5 | 3 | 32 |
| 8H | 3.0 | 10 | 15 | 5 | 5 | 3 | 32 |
| 9H | 3.0 | 15 | 10 | 5 | 5 | 3 | 32 |
| 10H | 3.0 | 15 | 15 | 5 | 5 | 3 | 32 |
| 11H | 3.0 | 30 | 10 | 5 | 5 | 3 | 32 |
| 12H | 3.0 | 30 | 15 | 5 | 5 | 3 | 32 |

Although the coupler for coupling the first arm 20, 120 and the second arm 30, 130 in the applicators 10, 100 described above with reference to FIGS. 1-9 is primarily illustrated in the drawings and described herein in the context as including both the guide 40, 140 and the locking device 50, 150, the applicators 10, 100 are not limited to such construction. Rather, in alternative embodiments, the coupler for coupling the first arm 20, 120 and the second arm 30, 130 can include only one of the guide 40, 140 and the locking device 50, 150.

Radiation sources that can be utilized in combination with the various applicator 10, 100 embodiments disclosed herein include, by way of non-limiting example, iridium-192 (Ir-192), cesium-137 (Cs-137), or cobalt-60 (Co-60) seeds that, in some cases, may be carried by a flexible wire, catheter, needle, or other carrying device suitable for insertion into the internal channels of the applicator 10, 100. In some embodiments and implementations, a radiation source loaded into the applicator 10, 100 may be arranged in the form of a train of radiation seeds. Loading techniques which may be employed to load a radiation source into the various embodiments of the applicators 10, 100 disclosed herein are readily known within the art. The combination of an applicator 10, 100 with a radiation source and/or device for carrying a radiation source can be characterized as an applicator assembly.

In some embodiments and implementations, a radiation source may be loaded into the various embodiments of the applicators 10, 100 disclosed herein via an afterloading machine, which, in some embodiments and implementations, may be computer-controlled. In some embodiments and implementations, an image guidance system, such as a computed tomography (CT) system, magnetic resonance imaging (MRI) system, and/or ultrasound system may be employed to facilitate guidance of the applicators 10, 100 to a desired location within an individual in need of brachytherapy. Treatment planning software can also be utilized in the treatment of an individual in need of brachytherapy to assist in the calculation of dose distribution and optimized source positioning for treatment of an individual.

Accordingly, the various applicator 10, 100 embodiments disclosed herein can be utilized as part of a larger system that includes one or more of: an afterloading machine that assists in the deposit of a radiation source into the internal channels of the applicator 10, 100; an image guidance system; and/or treatment planning software stored in a computer-readable medium and executed by one or more processors.

The various components of the applicator embodiments disclosed herein are preferably constructed of medical-grade materials. In this regard, and in some embodiments, the materials utilized to construct the components of the various applicator 10, 100 embodiments comply with International Organization Standardization (ISO) 10993. In various embodiments, the first arm 20, 120, the second arm 30, 130, the locking device 50, 150, and the conduit 160 may be constructed, in whole or in part, of a metal material, such as, by way of non-limiting example, titanium, stainless steel, cobalt-chromium, tantalum, platinum, magnesium, and combinations thereof. In some embodiments, the first arm 20, 120, the second arm 30, 130, the locking device 50, 150, and the conduit 160 is constructed of titanium. In some embodiments, the first arm 20, 120 and the second arm 30, 130 are of a rigid construction. In various embodiments, the guide 40, 140 may be constructed, in whole or in part, of a plastic material, such as, by way of non-limiting example, acetal copolymer (POM-C), acrylic (PMMA), polycarbonate, polyether ether ketone (PEEK), polypropylene (PP), polyphenylsulfone (PPSU), ultra-high molecular weight polyethylene, polytetrafluoroethylene (PTFE), polysulfone (PSU), polyphenylene sulfide (PPS), polyvinylidene fluoride (PVDF), polyetherimide (PEI), polyphenylene oxide (PPO), derlin, plastics from FormLabs® BioMed series of compatible plastics, and combinations thereof. In some embodiments, some or all the components of the applicator 10, 100 may be constructed, in whole or in part, of a radiopaque material or combination of radiopaque materials.

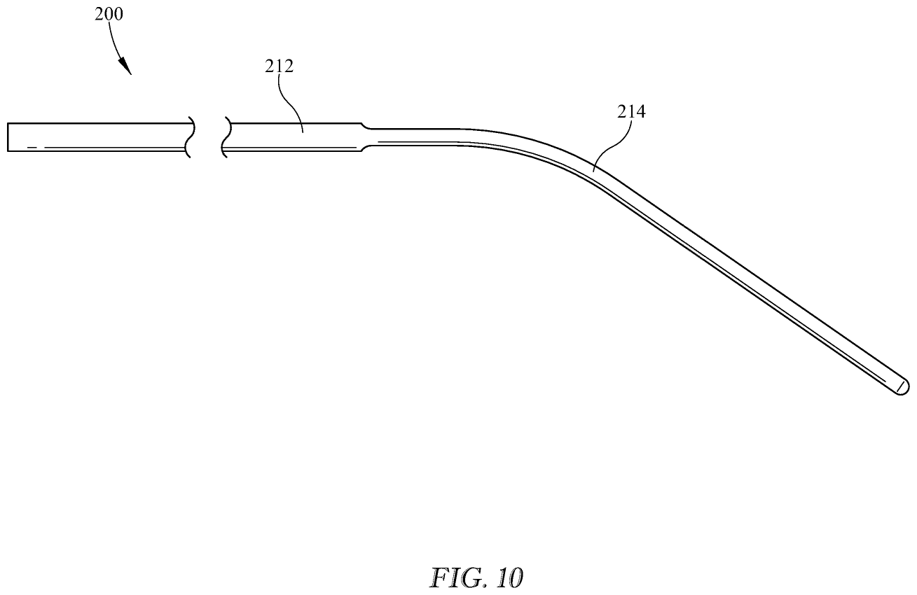

Referring now to FIG. 10, a side view of an alternative arm 200 construction that can be utilized in the applicators 10, 100 is provided. The arm 200 in this embodiment is of the same construction as the second arm 30, 130 describe above, except that the outer diameter of the handle 212 is larger than the outer diameter of the tandem 214. In some embodiments, the diameter of the handle 212 may be about 6 mm while the diameter of the tandem 214 is about 3 mm. In some embodiments, the diameter of the internal channel (not shown) collectively defined by the handle 212 and the tandem 214 is the same throughout its length despite the handle 212 and the tandem 214 being of different diameters. The arm 200 embodiment shown in FIG. 10 can be used in place of the second arm 30, 130 in alternative embodiments of the applicators 10, 100 described above with reference to FIGS. 1-9. Additionally or alternatively, an arm of similar construction to the arm 200 embodiment shown in FIG. 10, but with the curvature characteristics of the first arm 20, 120 described above can be used in place of the first arm 20, 120 in alternative embodiments of the applicators 10, 100 described above with reference to FIGS. 1-9.

Although the applicators 10, 100 are sometimes disclosed herein in the context of being utilized for the treatment of a particular type of cancer, it is appreciated that the disclosed applicators 10, 100 may find utility in the treatment of other types of cancer. In various implementations, the applicators 10, 100 disclosed herein may be utilized in high-dose rate or low-dose rate brachytherapy treatments.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. An intracavitary applicator for radiation oncology, comprising:
a first arm, including
a first handle defining a proximal end of the first arm, and
a first tandem defining a distal end of the first arm, the first tandem configured to be inserted in a uterine cavity of an individual, wherein the first handle and the first tandem collectively define a first internal channel with an open proximal end and a closed distal end, the first internal channel configured to receive and guide a radiation source from the proximal end of the first arm toward the distal end of the first arm; and
a second arm coupled to the first arm, the second arm including
a second handle defining a proximal end of the second arm, and
a second tandem defining a distal end of the second arm, the second tandem configured to be inserted in the uterine cavity of the individual, wherein the second handle and the second tandem collectively define a second internal channel with an open proximal end and a closed distal end, the second internal channel configured to receive and guide the radiation source from the proximal end of the second arm toward the distal end of the second arm;
wherein a portion of the first internal channel defined by the first tandem and a portion of the second internal channel defined by the second tandem each curve along multiple planes.

2. The intracavitary applicator of claim 1, wherein the portion of the first internal channel defined by the first tandem and the portion of the second internal channel defined by the second tandem each curve along two planes that are perpendicular to each other.

3. The intracavitary applicator of claim 2, wherein the portion of the first internal channel defined by the first tandem and the portion of the second internal channel defined by the second tandem are each oriented to curve along a sagittal plane and a coronal plane when the first tandem and the second tandem are inserted in the uterine cavity of the individual.

4. The intracavitary applicator of claim 1, wherein a portion of the first tandem is oriented at an angle of about 10 degrees to about 30 degrees relative to the first handle, and wherein a portion of the second tandem is oriented at an angle ranging from about 10 degrees to about 30 degrees relative to the second handle.

5. The intracavitary applicator of claim 1, wherein a portion of the first tandem is oriented at an angle of about 10 degrees to about 15 degrees relative to the first handle, and wherein a portion of the second tandem is oriented at an angle ranging from about 10 degrees to about 15 degrees relative to the second handle.

6. The intracavitary applicator of claim 1, wherein the first tandem and the second tandem each have a length ranging from about 6 centimeters to about 13 centimeters.

7. The intracavitary applicator of claim 1, wherein the first tandem and the second tandem each include a first curved portion and a second curved portion, and wherein the second curved portion of the first tandem and the second curved portion of the second tandem curve along a common plane in opposite directions when the first curved portion of the first tandem and the first curved portion of the second tandem are oriented to curve in the same direction along parallel planes.

8. The intracavitary applicator of claim 1, and further comprising:
a coupler for coupling the first arm and the second arm, the coupler including at least one of
a guide including a first internal channel in which the first arm is slidably received and a second internal channel in which the second arm is slidably received, and
a locking device configured to be selectively manipulated to hold the first arm and the second arm in a fixed position.

9. The intracavitary applicator of claim 8, wherein the coupler includes the guide, and wherein the guide has a diameter of about 2.0 centimeters to about 3.0 centimeters.

10. The intracavitary applicator of claim 8, wherein the coupler includes a third internal channel configured to receive the radiation source.

11. The intracavitary applicator of claim 10, and further comprising:
a conduit attached to the guide, the conduit being configured to direct the radiation source from outside of the guide into the third internal channel.

12. The intracavitary applicator of claim 10, wherein the third channel forms a ring.

13. The intracavitary applicator of claim 1, wherein a diameter of the first handle is larger than a diameter of the first tandem, and wherein a diameter of the second handle is larger than a diameter of the second tandem.

14. An intracavitary applicator for radiation oncology, comprising:
a first arm, including
a first handle defining a proximal end of the first arm, and
a first tandem defining a distal end of the first arm, the first tandem configured to be inserted in a uterine cavity of an individual, wherein the first handle and the first tandem collectively define a first internal channel with an open proximal end and a closed distal end, the first internal channel configured to receive and guide a radiation source from the proximal end of the first arm toward the distal end of the first arm;
a second arm, including
a second handle defining a proximal end of the second arm, and
a second tandem defining a distal end of the second arm, the second tandem configured to be inserted in the uterine cavity of the individual, wherein the second handle and the second tandem collectively define a second internal channel with an open proximal end and a closed distal end, the second internal channel configured to receive and guide the radiation source from the proximal end of the second arm toward the distal end of the second arm; and
a coupler coupling the first arm and the second arm, the coupler including at least one of
a guide including a first internal channel in which the first arm is slidably received and a second internal channel in which the second arm is slidably received, and
a locking device configured to be selectively manipulated to hold the first arm and the second arm in a fixed position;
wherein the first tandem and the second tandem each include a first curved portion and a second curved portion, and wherein the second curved portion of the first tandem and the second curved portion of the second tandem curve along a common plane in opposite directions when the first curved portion of the first tandem and the first curved portion of the second tandem are oriented to curve in the same direction along parallel planes.

15. The intracavitary applicator of claim 14, wherein a portion of the first internal channel defined by the first tandem and a portion of the second internal channel defined by the second tandem each curve along two planes that are perpendicular to each other.

16. The intracavitary applicator of claim 14, wherein a first portion of the first tandem is oriented at an angle of about 10 degrees to about 30 degrees relative to the first handle, wherein a first portion of the second tandem is oriented at an angle of 10 degrees to about 30 degrees relative to the second handle, wherein a second portion of the first tandem is oriented at an angle ranging from about 10 degrees to about 15 degrees relative to the first handle, wherein a second portion of the second tandem is oriented at an angle of about 10 degrees to about 15 degrees relative to the second handle.

17. The intracavitary applicator of claim 14, wherein the coupler includes the guide, and wherein the guide further includes a third internal channel configured to receive the radiation source.

18. The intracavitary applicator of claim 17, and further comprising:
a conduit attached to the guide, the conduit being configured to direct the radiation source from outside of the guide into the third internal channel.
